(19) United States
(12) Patent Application Publication
(10) Pub. No.: US 2022/0327425 A1
(43) Pub. Date: Oct. 13, 2022

(54) PEPTIDE MUTATION POLICIES FOR TARGETED IMMUNOTHERAPY

(71) Applicant: NEC Laboratories America, Inc., Princeton, NJ (US)

(72) Inventors: Renqiang Min, Princeton, NJ (US); Hans Peter Graf, South Amboy, NJ (US); Ligong Han, Edison, NJ (US)

(21) Appl. No.: 17/711,658

(22) Filed: Apr. 1, 2022

Related U.S. Application Data
(60) Provisional application No. 63/170,727, filed on Apr. 5, 2021.

(51) Int. Cl. G06N 20/00

(52) U.S. Cl. CPC G06N 20/00

(57) ABSTRACT

Methods and systems for training a machine learning model include embedding a state, including a peptide sequence and a protein, as a vector. An action, including a modification to an amino acid in the peptide sequence, is predicted using a presentation score of the peptide sequence by the protein as a reward. A mutation policy model is trained, using the state and the reward, to generate modifications that increase the presentation score.

This application claims priority to U.S. Provisional Patent Application No. 63/170,727, filed on Apr. 5, 2021, incorporated herein by reference in its entirety.

### BACKGROUND

### Technical Field

The present invention relates to immunotherapy, and, more particularly, to the modification of peptide sequences and prediction of modified peptide sequence binding affinities.

### Description of the Related Art

Peptide-MHC (Major Histocompatibility Complex) protein interactions are involved in cell-mediated immunity, regulation of immune responses, and transplant rejection. While computational tools exist to predict a binding interaction score between an MHC protein and a given peptide, tools for generating new binding peptides with new specified properties from existing binding peptides are lacking.

### SUMMARY

A method of training a machine learning model includes embedding a state, including a peptide sequence and a protein, as a vector. An action, including a modification to an amino acid in the peptide sequence, is predicted using a presentation score of the peptide sequence by the protein as a reward. A mutation policy model is trained, using the state and the reward, to generate modifications that increase the presentation score.

A method of developing treatments includes training a peptide mutation policy model to generate modifications to an input peptide based on a presentation score. A known peptide is sampled from a peptide library targeting a virus pathogen or tumor. The known peptide is mutated using the peptide mutation policy to generate a new peptide having an above-threshold presentation score by the MHC protein. A treatment is developed for a pathogen associated with the MHC protein using the new peptide.

A system for training a machine learning model includes a hardware processor and a memory. The memory stores a computer program, which, when executed by the hardware processor, causes the hardware processor to embed a state, including a peptide sequence and a protein, as a vector, to predict an action, including a modification to an amino acid in the peptide sequence, using a presentation score of the peptide sequence by the protein as a reward, and to train a mutation policy model, using the state and the reward, to generate modifications that increase the presentation score.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a diagram of a bond between a peptide and a major histocompatibility complex (MHC), in accordance with an embodiment of the present invention;

FIG. 2 is a block/flow diagram of a method of developing a treatment for a patient using mutated peptides based on peptides that correspond to a pathogen or tumor, in accordance with an embodiment of the present invention;

FIG. 3 is a block/flow diagram of a method of training a peptide mutation policy model using reinforcement learning, in accordance with an embodiment of the present invention;

FIG. 4 is a block diagram of a computing device that can train a peptide mutation policy model using reinforcement learning and that can mutate peptides using such a model, in accordance with an embodiment of the present invention;

FIG. 5 is a diagram of a neural network architecture that can be used as part of a machine learning model, in accordance with an embodiment of the present invention; and

FIG. 6 is a diagram of a deep neural network architecture that can be used as part of a machine learning model, in accordance with an embodiment of the present invention;

### DETAILED DESCRIPTION OF PREFERRED EMBODIMENTS

Interactions between peptides and major histocompatibility complexes (MHCs) play a role in cell-mediated immunity, regulation of immune responses, and transplant rejection. Prediction of peptide-protein binding helps guide the search for, and design of, peptides that may be used in vaccines and other medicines. Given a library of known peptides, new peptide sequences can be generated using mutation policies. The resulting mutated peptides may be within a threshold number of amino acid differences from the library of peptides. When the library of peptides is derived from a particular pathogen, such as a virus or tumor sample, the mutated peptides can be used to target the specific pathogen or tumor. This makes it possible to, for example, identify and target a specific cancer for an individual.

Thus, given a particular genome (e.g., sequenced from a virus or tumor cell), peptide sequences may be extracted to generate a library of peptides that uniquely identifies the pathogen. By targeting this library, peptides can be generated that bind to MHCs that are present on cell surfaces, so that immune responses can be triggered to kill the pathogen or tumor cells.

Toward that end, a deep neural network may be trained using a training dataset to predict a peptide presentation score given an MHC allele sequence and a peptide sequence. The peptide presentation score may be, e.g., a combination of peptide-MHC binding affinity and an antigen processing score.

Based on the trained peptide presentation model, deep reinforcement learning may be used to generate peptides with high presentation scores. Mutation policies may keep these generated peptides close to a provided target peptide library. The pretrained presentation score prediction model may be used to define reward functions starting from random peptides. The deep reinforcement learning system may be trained to learn good peptide mutation policies by transforming a given random peptide into a peptide with a high presentation score.

Batches of peptides may be randomly sampled from the target library and a mutation policy network can be used to mutate the peptides in the target library. The mutation process may stop when a mutated peptide reaches a threshold number of amino acid differences from a starting peptide, and the mutated peptide may be output. The policy network may be fine-tuned for the target library with a similarity constraint.

Each peptide in the target library may produce multiple mutated peptides that satisfy the similarity constraint. The output mutated peptides may be ranked, and the top-ranked mutated peptides may be used as drug candidates to target the pathogen or tumor for immunotherapy.

When applying a reinforcement learning system to this process, the “state” may be interpreted as being a given MHC allele sequence and peptide sequence, while the “action” may be interpreted as an edit to the peptide sequence. Such an edit may replace a current amino acid at a determined position of the peptide sequence with a new amino acid.

The amino acid sequences may be embedding using a convolutional layer and fully connected layers of a neural network model to generate an allele representation. A bi-directional long-short term memory (LSTM) layer may further process the amino acid embeddings to obtain a peptide representation. A deep policy network may then learn the conditional probability of the different actions may be learned given the state. At each time step, if the peptide presentation score of the mutated peptide based on an action is increased more than a threshold, it may be assigned a positive reward value, and otherwise it may be assigned a negative reward value.

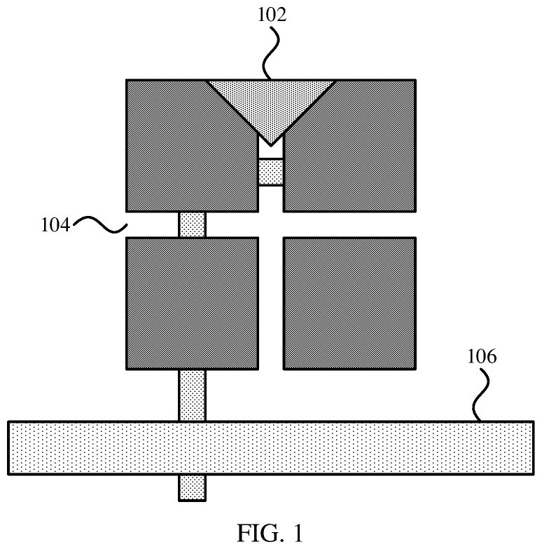

Referring now to FIG. 1, a diagram of a peptide-MHC protein bond is shown. A peptide 102 is shown as bonding with an MHC protein 104, with complementary two-dimensional interfaces of the figure suggesting complementary shapes of these three-dimensional structures. The MHC protein 104 may be attached to a cell surface 106.

An MHC is an area on a DNA strand that codes for cell surface proteins that are used by the immune system. MHC molecules are used by the immune system and contribute to the interactions of white blood cells with other cells. For example, MHC proteins impact organ compatibility when performing transplants and are also important to vaccine creation.

A peptide, meanwhile, may be a portion of a protein. When a pathogen presents peptides that are recognized by a MHC protein, the immune system triggers a response to destroy the pathogen. Thus, by finding peptide structures that bind with MHC proteins, an immune response may be intentionally triggered, without introducing the pathogen itself to a body. In particular, given an existing peptide that binds well with the MHC protein 104, a new peptide 102 may be automatically identified according to desired properties and attributes.

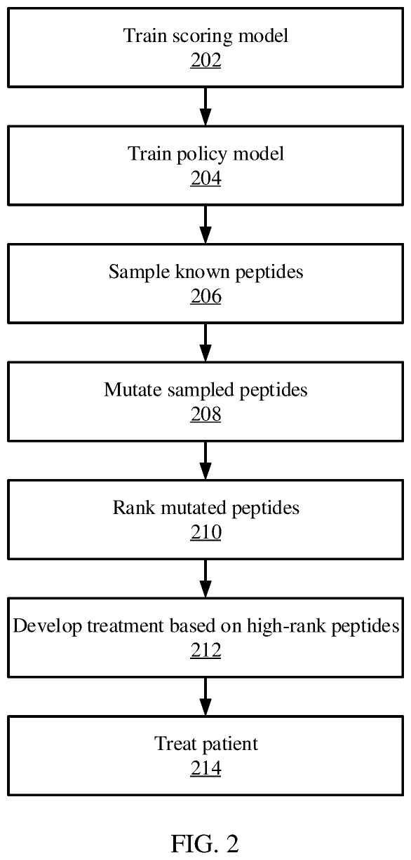

Referring now to FIG. 2, a method for treating an illness is shown. Block 202 trains a peptide scoring model, which accepts as input a peptide p and an MHC protein m and generates an output score r(p, m) that represents a binding affinity between the peptide p and the protein m, in particular representing the probability that the peptide p will be presented on a cell surface by the protein m. In some cases, the scoring model may be an off-the-shelf model, and so may come pre-trained. In some cases, the presentation score may be a composite score of an antigen processing prediction and a binding affinity prediction, where the former predicts a probability for a peptide to be delivered by the transporter associated with antigen processing protein complex into the endoplasmic reticulum, where the peptide can bind to MHC proteins.

Block 204 trains a mutation policy network, which will guide how peptide sequences are modified. As will be described in greater detail below, this policy network guides the reinforcement learning system, taking as an input a peptide and an MHC protein and outputting a modification or “mutation” of the peptide. The policy network selects the mutation with the goal of improving the presentation score of the mutated peptide to the MHC protein.

Block 206 samples a library of peptides relating to a pathogen in question. In some cases, this sampling may be performed randomly. In some cases, all of the peptides in the library may be evaluated. Block 208 then mutates the sampled peptides according to the mutation policy network, generating new mutated peptides that differ from the sampled peptides by, e.g., at most a predetermined number of amino acids. Block 210 ranks these mutated peptides according to their presentation score, with better bindings corresponding to higher ranks.

Having identified mutated peptides that bind well to the MHC protein of the pathogen, block 212 generates a treatment based on the peptides. Block 214 then treats a patient using the developed treatment, for example by administering a drug that includes the identified peptides, which bind to the MHC protein of the pathogen and encourage the patient's immune system to target the pathogen.

Within this framework, a peptide may be represented as a sequence of amino acids p=<o₁, o₂, . . . , oₗ>, where o is one of a set of natural amino acids and l is the length of the sequence, for example ranging between 8 and 15. A reinforcement learning agent explores the peptide mutation environment for high-presentation peptide generation. Thus, given a pair of inputs (p, m), the reinforcement learning agent explores and exploits the peptide mutation environment by repeatedly mutating the peptide and observing the resulting presentation score. The agent thereby learns the mutation policy π(·) to iteratively mutate amino acids of any given peptide to generate a high presentation score. Thus, a peptide mutation environment and a mutation policy network are determined.

The peptide mutation environment enables the reinforcement learning agent to perform trial-and-error peptide mutations to gradually refine its mutation policy, through tuning the parameters of the mutation policy network. During learning, the reinforcement learning agent keeps mutating peptides and determining their presentation scores as a reward signal. The rewards help reinforce the agent's mutation behaviors, with those mutation behaviors that produce high presentation scores being encouraged.

The mutation environment includes a state space, an action space, and a reward function. The state includes the current mutated peptide and the MHC protein. The action and the reward represent the mutation action that may be taken by the reinforcement learning agent, resulting in a new presentation score for the mutated peptide, respectively.

The state of the environment may be defined as sₜ at a time t for a pair (p, m). The MHC protein may be represented as a pseudo-sequence, for example with thirty-four amino acids, each being in potential contact with the bound peptide within a distance of, e.g., 4.0 Å. With a peptide of length l and an MHC protein, the state sₜ may be represented as the tuple sₜ=(E^{p}, E^{m}), where E^{p} and E^{m} are the encoding matrices of the peptide and the MHC protein, respectively. The state s₀ may be initialized by sampling a peptide sequence from a library and using an MHC class I protein. During training, any appropriate peptide sequence and MHC protein may be used. The terminal state s_{T} may be defined as the state with a maximum time step T or having a presentation score greater than a predetermined threshold σ. When the terminal state s_{T} is reached, the mutation of the peptide may be halted.

A multi-discrete action space may be defined to optimize the peptide by replacing one amino acid with another. At a time t, given a peptide pₜ, the action for the reinforcement learning agent may be to determine the position of the amino acid oᵢ being replaced and then to predict a type of new amino acid for that position. The reward function guides the optimization of the reinforcement learning agent, where only the terminal states can receive rewards from the peptide mutation environment. The final reward may be determined as r(p_{T}, m), with the peptide p_{T} being in the terminal state s_{T}.

To learn the mutation policy in block 204, the reinforcement learning agent learns to mutate amino acids in an input peptide sequence, one amino acid at each step, with the goal of maximizing the presentation score of the mutated peptide. Both the peptide and the MHC protein may be encoded into a distributed embedding space, and then a mapping between the embedding space and the mutation policy may be learned by a gradient descent optimization.

Multiple encoding methods may be used to represent the amino acids within the peptide sequences and the MHC proteins. Each amino acid may be represented by concatenating encoding vectors e^{B} from a block substitution matrix (BLOSUM), e^{O} from a one-hot matrix, and e^{D} from a learnable embedding matrix. Thus, e=e^{B}⊕e^{O}⊕e^{D} where e∈ [symbol] ^{d} (d=B+O+D). This achieves good binding prediction performance on peptide-MHC proteins. The encoding matrices E^{p} and E^{m} of the peptide p and the MHC protein m may then be represented as E^{p}={e₁; . . . ; eₗ}∈ [symbol] ^{l×d} and E^{m}={e₁; . . . ;e_{M}}∈ [symbol] ^{M×d}, respectively, with M being a number of available amino acids.

Each amino acid oᵢ in a peptide sequence p may be embedded into a continuous latent vector hᵢ using, for example, a one-layer bidirectional LSTM as:

\vec{h}ᵢ,\vec{c}ᵢ=LSTM(eᵢ,\vec{h}ᵢ₋₁,\vec{c}ᵢ₋₁,\vec{W}^{p})

[symbol]

ᵢ, [symbol] ᵢ=LSTM(eᵢ, [symbol] ᵢ₊₁, [symbol] ᵢ₊₁, [symbol] ^{p})

hᵢ=\vec{h}ᵢ⊕ [symbol] ᵢ

where [symbol] and \vec{h} are hidden state vectors of the i^{th} amino acid, \vec{c} and [symbol] are memory cell states of the i^{th} amino acid, \vec{h}₀, [symbol] ₗ, \vec{c}₀, and [symbol] ₗ are initialized with random values, and \vec{W}^{p} and [symbol] ^{p} are learnable parameters of the LSTM in the forward and backward direction, respectively. The embedding of the peptide sequence may be defined as the concatenation of hidden vectors at two ends: h^{p}=\vec{h}ₗ⊕ [symbol] ₀.

To embed an MHC protein into a continuous latent vector, the encoding matrix E^{m} is flattened into a vector m. The continuous latent embedding h^{m} may be learned as:

h^{m}=W₁^{m}ReLU(W₂^{m}m)

where ReLU(·) is a rectified linear unit activation function and Wₗ^{m}(l=1, 2) are learnable parameter matrices.

At each time step t, the peptide sequence pₜ may be optimized by predicting the mutation of one amino acid with the latent embeddings h^{p}^{ₜ} and h^{m}. Specifically, the amino acid oᵢ may be selected from pₜ as the amino acid to be replaced. For each amino acid oᵢ in the peptide sequence, the score of the replacement may be predicted as:

f^{c}(oᵢ)=(w^{c})^{T}(ReLU(W₁^{c}hᵢ+W₂^{c}h^{m})

where hᵢ is the hidden latent vector of oᵢ, and w^{c} and Wₗ^{c} are the learnable vector and matrices, respectively. The likelihood of replacing amino acid oᵢ with another amino acid can be measured by looking at its context in hᵢ and the MHC protein h^{m}. The amino acid to be replaced may be determined by sampling from the distribution with normalized scores. The type of amino acid that replaces oᵢ may be determined as:

f^{d}(o)=softmax(W₁^{d}×ReLU(W₂^{d}hᵢ+W₃^{d}h^{m})

where Wₗ^{d} (l=1, 2, 3) are learnable matrices and where softmax(·) converts a twenty-dimensional vector into probabilities over the twenty amino acid types. The amino acid type may then be determined by sampling from the distribution of probabilities of amino acid types, excluding the original amino acid type oᵢ.

The objective function for learning the mutation policy may be defined as:

$${max}_{θ} {L}^{CLIP} ( θ ) = {t}_{} [ min ( {r}_{t} ( θ ) {\hat{A}}_{t} , clip ( {r}_{t} ( θ ) , 1 - ϵ , 1 + ϵ ) {\hat{A}}_{t} ) ]$$

where [symbol] is an expectation with respect to a time step t (e.g., the average over all time steps), θ is the set of learnable parameters of the policy network and

$${r}_{t} ( θ ) = \frac{{π}_{θ} ( {a}_{t} \vert {s}_{t} )}{{π}_{{θ}_{o l d}} ( {a}_{t} \vert {s}_{t} )}$$

is the probability ratio between the action under current policy π_{θ} and the action under the previous policy π_{θ}_{_{old}}. The ratio rₜ(θ) is clipped to avoid moving rₜ outside the interval [1−ϵ, 1+ϵ]. The term Âₜ is the advantage at time step t, computed with a generalized advantage estimator, measuring how much better the selected actions are than others on average:

Âₜ=δₜ+(γλ)δₜ₊₁+ . . . +(γλ)^{T-t+1}δ_{T-1}

where γ∈(0,1) is a discount factor determining the importance of future rewards, δₜ=rₜ+γV(sₜ₊₁)−V(sₜ) is the temporal difference error, V(sₜ) is a value function, and λ∈(0,1) is a parameter used to balance the bias and variance of V(sₜ).

The value function V(sₜ) may use a multi-layer perceptron to predict the future return of current state sₜ from the MHC embedding h^{m} and the peptide embedding h^{p}. The objective function of V(·) may be defined as:

$${min}_{θ} {L}^{V} ( θ ) = {t}_{} [ {( V ( {s}_{t} ) - {\hat{R}}_{t} )}^{2} ]$$

where \hat{R}ₜ=Σᵢ₌ₜ₊₁^{T}γ^{i-t}rᵢ is a rewards-to-go value. Because only the final rewards are used (e.g., rᵢ=0∀i≠T), \hat{R}ₜ may be calculated as \hat{R}ₜ=γ^{T-t}r_{T}. The entropy regularization loss H(θ) may also be used to encourage exploration of the policy.

To stabilize the training and to improve performance, an expert policy πₑₚₜ may be derived from existing data. For each MHC protein m with sufficient binding peptide data, the amino acid distributions <p₁(o|m), p₂(o|m), . . . , pₗ(o|m)> of peptides with length l may be determined. Given a peptide p, the position I may be selected as follows:

$${p}_{ept}^{c} ( p , m ) = {argmax}_{i} ( {p}_{i} ( o = {\hat{o}}_{i} ) - {p}_{i} ( o = {o}_{i} \vert m ) )$$

where ôᵢ is the most popular amino acid on position i. In other words,

$${p}_{i} ( o = {\hat{o}}_{i} \vert m ) = {max}_{o} ( {p}_{i} ( o \vert m ) ) .$$

After determining the position, the amino acid can be sampled from the distribution oᵢ′˜pᵢ(o|m). For an MHC protein without experimental data, the distances can be calculated with all of the MHCs with data, for example using a block substitution matrix, and actions can be sampled from the amino acid distributions with the most similar MHC.

The expert policy can be used to pre-train the policy network. The objective function for pre-training can minimize the following cross-entropy loss:

$${min}_{θ} {L}^{PRE} ( θ ) = {s \sim S}_{} [ {i \sim {π}_{ept}^{c}}_{} [ log ( {π}_{θ}^{c} ( i \vert s ) ) ] + {o \sim {π}_{ept}^{d}}_{} [ log ( {π}_{θ}^{d} ( o \vert s ) ) ] ]$$

where S denotes the state space, π_{θ}^{c} and π_{θ}^{d} are, respectively, parameterized by f^{c} and f^{d}, which are the policy networks for selecting the position and the amino acid for mutation. In addition to pre-training the policy network, actions can be sampled at the beginning of training using the expert policy, and the trajectories can be used with expert actions to update the policy network.

To increase the diversity of generated peptides, a non-deterministic policy can be used to produce diverse actions. Such a policy can increase the exploration over a large state space and can thereby find diverse good actions.

Entropy regularization can be included in the objective function to promote exploration. To explicitly enforce the policy's learning of diverse actions, a diversity-promoting experience buffer may be used to store trajectories that could result in qualified peptides. At each iteration, the visited state-action pairs of mutation trajectories for qualified peptides can be added to the buffer. The state-action pairs may be maintained with infrequent actions, and those with frequent actions can be removed to ensure that the buffer is not dominated by the frequent actions. A batch of state-action pairs with infrequent actions can be sampled from the buffer.

A cross-entropy loss L^{B} defined over the batch of state-action pairs with infrequent actions can then be included in the final objective function, to encourage the policy network to reproduce those infrequent actions that could induce high rewards:

$${min}_{θ} L ( θ ) = - {L}^{CLIP} ( θ ) + {α}_{1} {L}^{V} ( θ ) + {α}_{2} {L}^{B} ( θ ) + {α}_{3} H ( θ )$$

where H is the entropy of the policy network, and α₁, α₂, α₃ are predetermined coefficients.

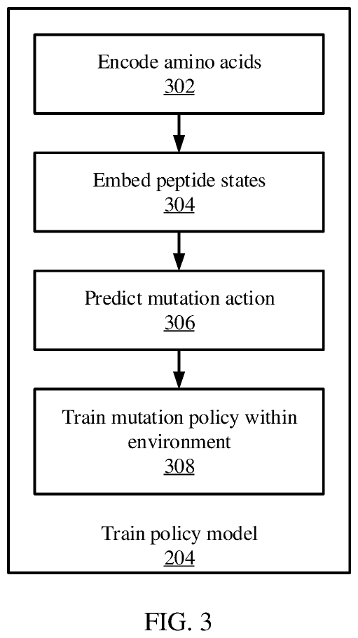

Referring now to FIG. 3, additional detail on the training of the policy model in block 204 is shown. Block 302 encodes amino acids, for example using a mixture of different encodings. Block 304 embeds the peptide sequences (e.g., of a library) using a bidirectional LSTM and embeds the MHC protein into a continuous latent vector using a flattened encoding matrix.

Block 306 uses a current mutation policy to predict the reward of a mutation action. The action may be selected as described above, and the reward may be calculated based on a binding strength indicated by a pre-trained presentation score model. Block 308 may train the mutation policy based on the rewards, so that the mutation policy indicates mutation actions that tend to produce the highest rewards.

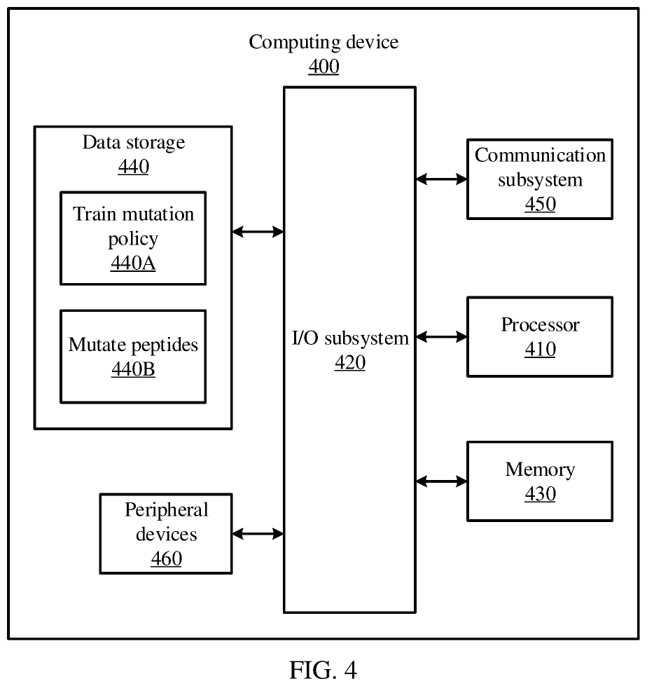

Referring now to FIG. 4, an exemplary computing device 400 is shown, in accordance with an embodiment of the present invention. The computing device 400 is configured to perform classifier enhancement.

The computing device 400 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 400 may be embodied as a one or more compute sleds, memory sleds, or other racks, sleds, computing chassis, or other components of a physically disaggregated computing device.

As shown in FIG. 4, the computing device 400 illustratively includes the processor 410, an input/output subsystem 420, a memory 430, a data storage device 440, and a communication subsystem 450, and/or other components and devices commonly found in a server or similar computing device. The computing device 400 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 430, or portions thereof, may be incorporated in the processor 410 in some embodiments.

The processor 410 may be embodied as any type of processor capable of performing the functions described herein. The processor 410 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 430 may be embodied as any type of volatile or non-volatile memory or data storage capable of performing the functions described herein. In operation, the memory 430 may store various data and software used during operation of the computing device 400, such as operating systems, applications, programs, libraries, and drivers. The memory 430 is communicatively coupled to the processor 410 via the I/O subsystem 420, which may be embodied as circuitry and/or components to facilitate input/output operations with the processor 410, the memory 430, and other components of the computing device 400. For example, the I/O subsystem 420 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 420 may form a portion of a system-on-a-chip (SOC) and be incorporated, along with the processor 410, the memory 430, and other components of the computing device 400, on a single integrated circuit chip.

The data storage device 440 may be embodied as any type of device or devices configured for short-term or long-term storage of data such as, for example, memory devices and circuits, memory cards, hard disk drives, solid state drives, or other data storage devices. The data storage device 440 can store program code 440A for training a mutation policy model and program code 440B for mutating peptide sequences according to a mutation policy model. The communication subsystem 450 of the computing device 400 may be embodied as any network interface controller or other communication circuit, device, or collection thereof, capable of enabling communications between the computing device 400 and other remote devices over a network. The communication subsystem 450 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 400 may also include one or more peripheral devices 460. The peripheral devices 460 may include any number of additional input/output devices, interface devices, and/or other peripheral devices. For example, in some embodiments, the peripheral devices 460 may include a display, touch screen, graphics circuitry, keyboard, mouse, speaker system, microphone, network interface, and/or other input/output devices, interface devices, and/or peripheral devices.

Of course, the computing device 400 may also include other elements (not shown), as readily contemplated by one of skill in the art, as well as omit certain elements. For example, various other sensors, input devices, and/or output devices can be included in computing device 400, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 400 are readily contemplated by one of ordinary skill in the art given the teachings of the present invention provided herein.

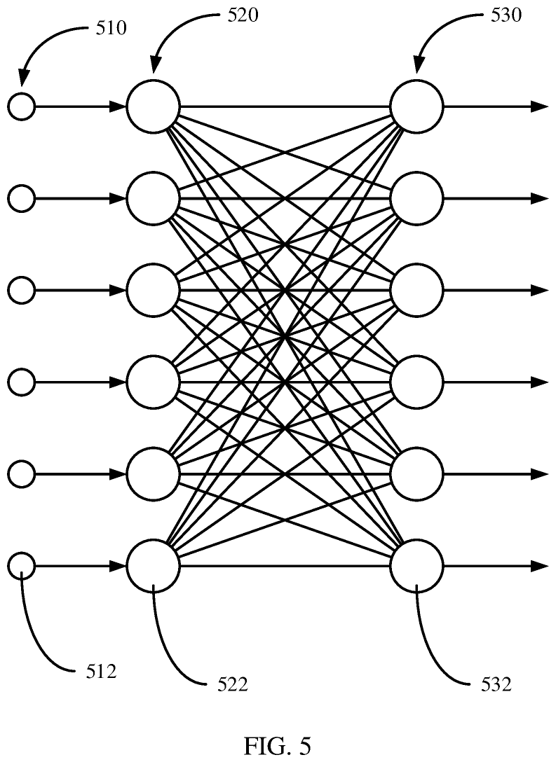

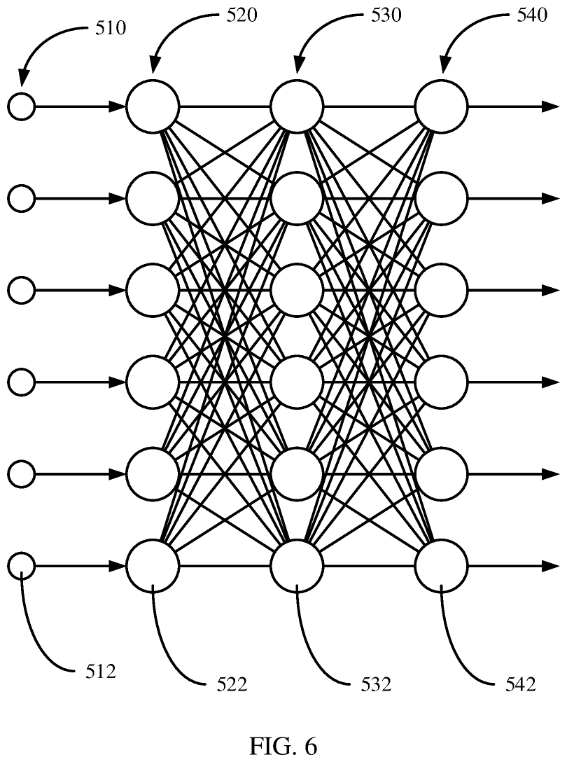

Referring now to FIGS. 5 and 6, exemplary neural network architectures are shown, which may be used to implement parts of the present models. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the inputted data belongs to each of the classes can be outputted.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 520 of source nodes 522, and a single computation layer 530 having one or more computation nodes 532 that also act as output nodes, where there is a single computation node 532 for each possible category into which the input example could be classified. An input layer 520 can have a number of source nodes 522 equal to the number of data values 512 in the input data 510. The data values 512 in the input data 510 can be represented as a column vector. Each computation node 532 in the computation layer 530 generates a linear combination of weighted values from the input data 510 fed into input nodes 520, and applies a non-linear activation function that is differentiable to the sum. The exemplary simple neural network can perform classification on linearly separable examples (e.g., patterns).

A deep neural network, such as a multilayer perceptron, can have an input layer 520 of source nodes 522, one or more computation layer(s) 530 having one or more computation nodes 532, and an output layer 540, where there is a single output node 542 for each possible category into which the input example could be classified. An input layer 520 can have a number of source nodes 522 equal to the number of data values 512 in the input data 510. The computation nodes 532 in the computation layer(s) 530 can also be referred to as hidden layers, because they are between the source nodes 522 and output node(s) 542 and are not directly observed. Each node 532, 542 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the inputted data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 532 in the one or more computation (hidden) layer(s) 530 perform a nonlinear transformation on the input data 512 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method of training a machine learning model, comprising:
embedding a state, including a peptide sequence and a protein, as a vector;
predicting an action, including a modification to an amino acid in the peptide sequence, using a presentation score of the peptide sequence by the protein as a reward; and
training a mutation policy model, using the state and the reward, to generate modifications that increase the presentation score.

2. The method of claim 1, further comprising training a scoring model that generates the presentation score.

3. The method of claim 2, wherein the presentation score represents a combination of a peptide-protein binding affinity and an antigen processing score.

4. The method of claim 1, wherein training the mutation policy includes minimizing a loss function that includes a clipping term, a reward term, and an exploration term.

5. The method of claim 1, wherein embedding the state is performed using a bi-directional long-short term memory (LSTM) neural network.

6. The method of claim 1, wherein the protein is a major histocompatibility complex (MHC) protein.

7. A computer-implemented method of developing treatments, comprising:
training a peptide mutation policy model to generate modifications to an input peptide based on a presentation score;
sampling a known peptide from a peptide library targeting a virus pathogen or tumor;
mutating the known peptide using the peptide mutation policy to generate a new peptide having an above-threshold presentation score by the MHC protein; and
developing a treatment for a pathogen associated with the MHC protein using the new peptide.

8. The method of claim 7, wherein the presentation score represents a combination of a peptide-protein binding affinity and an antigen processing score.

9. The method of claim 7, wherein training the mutation policy includes minimizing a loss function that includes a clipping term, a reward term, and an exploration term.

10. The method of claim 7, further comprising deriving the known peptide from a pathogen or tumor.

11. The method of claim 10, wherein the pathogen is a virus.

12. The method of claim 10, wherein the pathogen is from a tumor.

13. The method of claim 10, further comprising treating a person for the pathogen using the developed treatment.

14. The method of claim 7, wherein sampling the known peptide is repeated for a library of known peptides and mutating the known peptide is repeated for the library of known peptides, and further comprising ranking mutated peptides according to respective presentation scores.

15. A system for training a machine learning model, comprising:
a hardware processor; and
a memory that stores a computer program, which, when executed by the hardware processor, causes the hardware processor to:
embed a state, including a peptide sequence and a protein, as a vector;
predict an action, including a modification to an amino acid in the peptide sequence, using a presentation score of the peptide sequence by the protein as a reward; and
train a mutation policy model, using the state and the reward, to generate modifications that increase the presentation score.

16. The system of claim 15, wherein the computer program further causes the hardware processor to score train a scoring model that generates the presentation score.

17. The system of claim 16, wherein the presentation score represents a combination of a peptide-protein binding affinity and an antigen processing score.

18. The system of claim 15, wherein the computer program causes the hardware processor to train the mutation policy by minimizing a loss function that includes a clipping term, a reward term, and an exploration term.

19. The system of claim 15, wherein the computer program causes the hardware processor to embed the state using a bi-directional long-short term memory (LSTM) neural network.

20. The system of claim 15, wherein the protein is a major histocompatibility complex (MHC) protein.
